(19) United States
(12) Patent Application Publication
(10) Pub. No.: US 2025/0235222 A1
(43) Pub. Date: Jul. 24, 2025

(54) MEDICAL DEVICES WITH PASSIVE AND ACTIVE DRAINS

(71) Applicant: BOSTON SCIENTIFIC SCIMED, INC., Maple Grove, MN (US)

(72) Inventors: MICHAEL S H CHU, Brookline, MA (US); SACHA TANG, Tygnsborough, MA (US); MANUEL B. TEIXEIRA, Boston, MA (US)

(73) Assignee: BOSTON SCIENTIFIC SCIMED, INC., Maple Grove, MN (US)

(21) Appl. No.: 19/035,734

(22) Filed: Jan. 23, 2025

Related U.S. Application Data
(60) Provisional application No. 63/624,579, filed on Jan. 24, 2024.

(51) Int. Cl. A61B 17/22; A61B 17/00

(52) U.S. Cl. CPC A61B 17/22004; A61B 2017/00128; A61B 2017/22079; A61B 2217/007

(57) ABSTRACT

An illustrative medical device may include a sheath defining a lumen having an inner surface and an outer surface and a tubular elongate member extending through the lumen. The medical device may include an outlet channel in a portion of the lumen and a drain valve configured to couple the outlet channel to a drain conduit, wherein the drain valve includes a movable member that is configured to move between a first position and a second position, and wherein the drain valve is configured to passively drain material from the outlet channel when the movable member is in the first position and actively drain material from the outlet channel when the movable member is in the second position.

### CROSS REFERENCE TO RELATED APPLICATIONS

This application claims the benefit of and priority to U.S. Provisional Patent Application Ser. No. 63/624,579, filed on Jan. 24, 2024, the disclosure of which is incorporated herein by reference.

### FIELD

The present disclosure relates to medical devices, systems, and methods with passive and active drains. More particularly, the present disclosure relates to medical devices, systems, and methods with passive and active drains that are suitable to remove solid materials such as a bodily mass and/or portions thereof, from a patient's body cavity.

### BACKGROUND

Various medical conditions involve the presence of undesired bodily masses, such as hard stones, soft stones, impacted stones, tissue, tumors, etc., which are removable via surgical intervention, such as to remedy the condition. The bodily masses may be physically disrupted and reduced in size (to facilitate removal thereof) by a fragmenting device configured to direct energy, such as electric, hydraulic, laser, mechanical, ultrasound, etc., energy, at the bodily mass. For instance, various devices, such as lasers and/or laser fibers, can be inserted into an anatomical structure located (e.g., an anatomical cavity, such as a kidney) at which an undesired bodily mass (e.g., a kidney stone) is located to fragment and/or to dust the bodily mass to a smaller size capable of being more readily removed. For example, lithotripsy devices may be used to direct pulsed light energy at kidney stones. The energy is converted into mechanical and thermal energy, such as in the form of a cavitation bubble associated with the occurrence of a shockwave. The energy (e.g., cavitation bubble) may help to fragment, disrupt, cauterize, cut, break up, pulverize, etc., the kidney stone(s). In some aspects, the fragmenting device is inserted and advanced to the bodily mass through a lumen (e.g., working channel) of a tubular elongate member (e.g., a medical scope such as a ureteroscope). Devices can be inserted through the lumen of the tubular elongate member and advanced to the remnants of the bodily mass (e.g., smaller sized stones and/or stone fragments) to remove the remnants. For instance, retrieval devices, such as retrieval baskets, may be advanced to the remnants to remove the remnants. However, it may be difficult and time consuming to capture fine-sized fragments (e.g., smaller than 3 mm). Flushing the dust particles or using a popcorning technique (e.g., breaking down fragments of the bodily mass by impacting the fragments against one another or another structure) can scatter fragments/particles, particularly if located within an anatomical cavity, making it more difficult and time consuming to remove the unwanted material from the patient's body. Moreover, once the materials are captured by a retrieval device, the removal process, when feasible, may be time consuming as well. Typically, the retrieval device, along with the tubular elongate member, may need to be entirely withdrawn from the human body each time remnants of the bodily mass are captured by a retrieval device, particularly if the fragments are too large to fit through the lumen of the tubular elongate member (e.g., working channel of the medical scope). The tubular elongate member and retrieval device are then reinserted, to capture and remove the next fragment. Repeated insertion and removal of the retrieval device and tubular elongate member becomes very time consuming and prolongs the procedure. Large bodily masses and/or fragments thereof may sometimes be too large to be readily removed and may clog removal devices.

Although aspiration devices may be used to retrieve the remnants of the bodily mass, the bodily mass must be dusted to a very small size to be aspirated through the typically small diameter (e.g., relative to the initial size of the bodily mass) of the aspiration device lumen. The aspiration devices may include a control aperture that is fluidically coupled to a suction port of the suction device. The control aperture may offer a manual pressure/suction control. For example, an operator may cover the control aperture (e.g., vent) with their finger (e.g., thumb) to prevent suction from being lost (e.g., increase suction) or may partially cover the control aperture to vary a magnitude of the suction. However, such approaches (e.g., which are reliant upon the position of a finger of an operator and/or that do not provide a visual indication of a degree of applied suction) may be prone to over or under pressurizing into an anatomical structure (e.g., an anatomical cavity, such as a kidney). Complications may occur if the delicate pressure balance within an anatomical structure is not carefully maintained. For instance, in the case of removal of materials from a kidney with irrigation and aspiration, high renal pelvic pressure may cause such complications as renal damage, fever, systemic inflammatory response syndrome, sepsis, and/or other adverse conditions.

Solutions to these and other challenges in the art would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a medical device is disclosed. The medical device comprising a sheath defining a lumen therethrough and having an inner surface and an outer surface; an outlet channel in a portion of the lumen; and a drain valve configured to couple the outlet channel to a drain conduit, wherein the drain valve includes a movable member that is configured to move between a first position and a second position, and wherein the drain valve is configured to: passively drain material from the outlet channel to the drain conduit when the movable member is in the first position; and actively drain material from the outlet channel to the drain conduit when the movable member is in the second position.

In some aspects, the drain valve is coupled to a sidearm extending from a body of the medical device.

In some aspects, the sidearm is a straight sidearm.

In some aspects, the drain valve includes a dispositioning mechanism to disposition the movable member to the first position.

In some aspects, the dispositioning mechanism is a spring.

In some aspects, the drain valve includes a visual flow indicator between the outlet channel and the drain conduit that is configured to indicate a flow rate of the material between the outlet channel and the drain conduit.

In some aspects, the visual flow indicator includes a paddlewheel.

In some aspects, the drain conduit is an individual drain conduit that is coupled to a suction source.

In some aspects, the drain valve includes a vent.

In some aspects, the movable member includes a movable cover disposed on an outer surface of a sidearm, wherein the cover has a length that is equal or greater than a length of the vent, and wherein the cover is configured to: expose the vent when the cover is in the first position; and block at least a portion of the vent when the cover is in the second position.

In some aspects, the movable member is a plunger with a width that is equal to or greater than a width of the vent, and wherein the plunger is configured to: expose the vent when the plunger is in the first position; and block at least a portion of the vent when the plunger is in the second position.

In some aspects, the drain conduit includes a passive drain conduit and an active drain conduit; the active drain conduit is coupled to a suction source and is configured to operate at a reduced pressure relative to atmospheric pressure; and the passive drain conduit is configured to operate at atmospheric pressure.

In some aspects, the drain valve is configured to actively drain the material, via the active drain conduit, at the reduced pressure when the movable member is in the second position.

In some aspects, the drain valve is configured to passively drain the material, via the passive drain conduit, at atmospheric pressure when the movable member is in the first position.

In some aspects, the drain valve includes a slot, wherein the slot is configured to receive the material from the outlet channel, the slot having a surface including an outlet, wherein the movable member is a plunger configured to: block an active drain path extending through the active drain conduit when the movable member is in a first position; and align the outlet with the active drain conduit when the movable member is in a second position.

In accordance with various principles of the present disclosure, a system for removing material from an anatomical site is disclosed. The system comprising a sheath defining a single lumen therethrough and having an inner surface and an outer surface; a tubular elongate member extending through the single lumen and defining a distal inlet channel therethrough fluidly couplable with a fluid source to irrigate the anatomical site; an outlet channel between the inner surface of the sheath and the outer surface of the tubular elongate member, wherein the outlet channel is couplable with a suction source to aspirate material from the anatomical site; and a drain valve configured to couple the outlet channel to a drain conduit, wherein the drain valve includes a movable member that is configured to move between a first position and a second position, and wherein the drain valve is configured to: passively drain material from the outlet channel to the drain conduit when the movable member is in the first position; and actively drain material from the outlet channel to the drain conduit when the movable member is in the second position.

In some aspects, the system further comprises a fragmenting device that extends through the tubular elongate member.

In some aspects, the material comprises a bodily mass or fragments thereof.

In accordance with various principles of the present disclosure, a system for removing material from an anatomical site is disclosed. The system comprising an outer sheath defining an outer lumen therethrough and having an inner surface and an outer surface; an inner sheath defining an inner lumen therethrough and having an inner surface and an outer surface; a tubular elongate member extending through the inner lumen and defining a distal inlet channel therethrough fluidly couplable with a fluid source to irrigate the anatomical site; a biasing member configured to maintain an outlet channel between the inner surface of the inner sheath and the outer surface of the tubular elongate member, wherein the outlet channel is couplable with a suction source to aspirate material from the anatomical site; a drain valve configured to couple the outlet channel to a drain conduit, wherein the drain valve includes a movable member that is configured to move between a first position and a second position, and wherein the drain valve is configured to: passively drain material from the outlet channel to the drain conduit when the movable member is in the first position; and actively drain material from the outlet channel to the drain conduit when the movable member is in the second position; and a visual flow indicator located between the outlet channel and the drain conduit, the visual flow indicator configured to indicate a flow rate of the material between the outlet channel and the drain conduit.

In some aspects, the system comprises a dilator insertable within at least one of the outer sheath or the inner sheath; and a sensor operatively associated with one of the inner sheath or the tubular elongate member, or insertable separate from the inner sheath and the tubular elongate member.

These and other features and advantages of the present disclosure will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but are intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 1000, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of a fragmenting and/or fragment-removing system including passive and active drains in accordance with aspects of the present disclosure.

FIG. 1A illustrates a detail view of detail area 1A-1A in FIG. 1.

FIG. 1B illustrates an end view along line 1B-1B in FIG. 1.

FIG. 1C is an end view along line 1C-1C in FIG. 1.

FIG. 1D is a cross-sectional view along line 1D-1D in FIG. 1.

FIG. 1E illustrates a detail view of detail area 1E-1N in FIG. 1.

FIG. 1F illustrates a detail view of an example of the detail area 1E-1N in FIG. 1.

FIG. 1G illustrates a detail view of an example of the detail area 1E-1N in FIG. 1.

FIG. 1H illustrates a detail view of an example of the detail area 1E-1N in FIG. 1.

FIG. 1I illustrates a detail view of an example of the detail area 1E-1N in FIG. 1.

FIG. 1J illustrates a detail view of an example of the detail area 1E-1N in FIG. 1.

FIG. 1K illustrates a detail view of an example of the detail area 1E-1N in FIG. 1.

FIG. 1L illustrates a detail view of an example of the detail area 1E-1N in FIG. 1.

FIG. 1M illustrates detail view of an example of the detail area 1E-1N in FIG. 1.

FIG. 1N illustrates a detail view of an example of the detail area 1E-1N in FIG. 1.

FIG. 2A illustrates an elevational view of an alternate arrangement of elements of a fragmenting and/or fragment-removing system with a drain valve in a first position in accordance with aspects of the present disclosure.

FIG. 2B illustrates an elevational view of an alternate arrangement of elements of a fragmenting and/or fragment-removing system with a drain valve in a second position in accordance with aspects of the present disclosure.

FIG. 3A illustrates an elevational view of an alternate arrangement of elements of a fragmenting and/or fragment-removing system with a drain valve in a first position in accordance with aspects of the present disclosure.

FIG. 3B illustrates an elevational view of an alternate arrangement of elements of a fragmenting and/or fragment-removing system with a drain valve in a second position in accordance with aspects of the present disclosure.

FIG. 3C illustrates a perspective view of an example of a movable member in FIG. 3A and FIG. 3B.

FIG. 3D illustrates an end view along line 3A-3A in FIG. 3A.

FIG. 4A illustrates an elevational view of an example of an embodiment of an arrangement of a dilator with a fragmenting and/or fragment-removing system formed in accordance with various principles of the present disclosure.

FIG. 4B illustrates a perspective view of the example of an embodiment of FIG. 4A.

FIG. 4C illustrates a top view of the example of an embodiment of FIG. 4A.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “passage” is not limited to a circular cross-section. As used herein, a “free end” or “distalmost end” of an element is a terminal end at which such element does not extend beyond. It will be appreciated that terms such as at or on or adjacent or along an end may be used interchangeably herein without intent to limit unless otherwise stated and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

In accordance with various principles of the present disclosure, devices, systems, and methods are described herein for removing bodily masses and/or fragmented bodily masses from an anatomical site. It will be appreciated that reference may be made interchangeably herein to terms such as (including other grammatical forms of terms such as) fragments, particles, pieces, segments, remnants, portions, parts, dust, etc., of a larger element, such as a bodily mass, without intent to limit unless otherwise specified. The devices, system, and method described herein are optionally also configured to fragment undesired bodily masses to be removed in accordance with various principles of the present disclosure. Various removal devices disclosed herein are adapted to manage different-sized fragments. The aspiration devices may be used in conjunction with a fragmenting device configured to fragment the bodily mass into particles which may be more readily removed by the aspiration device. The fragmenting device may break the bodily mass into smaller sized fragments and/or into even smaller sizes (approximately 1 mm in size/diameter) typically referenced in the art as dust. Dust particles that are smaller than 250 microns (μm) can be suspended in fluid (e.g., water), while fragments larger than 250 microns will quickly sink in fluid. Aspiration devices formed in accordance with various principles of the present disclosure may be adjustable in size to be capable of removing fragments of varying sizes. Alternatively or additionally, aspiration devices formed in accordance with various principles of the present disclosure may be configured to be used in cooperation with a fragmenting device, such as to facilitate operation of a fragmenting device. In some aspects, the fragmenting device includes outlet channels through which fragments of the bodily masses are suctioned or aspirated out of the patient, or at least away from the initial site of the bodily mass. In some aspects, the aspiration device may simply be formed as an aspiration or outlet channel.

In some aspects, aspiration devices formed in accordance with various principles of the present disclosure are configured to maintain the appropriate pressure/fluid volume and/or temperature within the anatomical structure from which the bodily mass is to be removed. For instance, if the bodily mass is being removed from an anatomical cavity (e.g., a kidney), suction applied to aspirate fragments of the bodily mass may reduce the pressure within the anatomical cavity. In accordance with various principles of the present disclosure, a fragmenting and/or fragment-removing system includes one or more inlet channels, such as irrigation channels, and an outlet channel (in some embodiments, one or more outlet channels). In some aspects, a distal inlet channel and a proximal inlet channel are provided. In some aspects, the position of the distal end of the outlet channel through which fragments are removed is adjustable/movable with respect to the distal inlet channel and/or the proximal inlet channel.

In accordance with various principles of the present disclosure, a fragmenting device capable of delivering sufficient energy to fragment a bodily mass, may be positioned at an anatomical site to fragment a bodily mass along with an aspiration device. As such, aspiration of fragments may be performed during use of the fragmenting device to aspirate fragments as they are being created by the fragmenting device. Moreover, the fragmenting device may be delivered through a medical scope such as a ureteroscope, with the outlet channel positioned to aspirate materials as the fragmenting device reduces the size of the bodily mass. As such, the devices, systems, and methods of the present disclosure may avoid the need to remove, reinsert, and reposition the medical scope and fragmenting device to aspirate materials from the treatment site, as may be required, even multiple times, by current devices, systems, and methods. Because the aspiration device of the present disclosure is adjustable, as noted above, the aspiration device may be adjusted to accommodate removal of large fragments which previously were too large to fit through previous aspiration devices. Moreover, because the adjustability of the aspiration device allows for removal of fragments which were too large to be removed by previous aspiration devices, particles may be removed adjacent or in the general vicinity of the fragmenting device and need not be flushed to another location for removal (e.g., to the ureteropelvic junction (UPJ) in the case of lithotripsy in a kidney) as may have been required by previous devices. As used herein, terms of proximity such “in the general vicinity of” or “adjacent” should be readily understandable by those of ordinary skill in the art as close enough to affect operation of a device or system. For instance, as described herein, an outlet channel in the general vicinity of a fragmenting device allows aspiration of materials quickly enough so as not interfere with fragmenting operations. For instance, aspiration channels adjacent a fragmenting device may clear the path between the fragmenting device and the bodily mass being fragmented thereby, as the fragmenting device is being operated, so that fragments created during such operation do not in turn interfere with further operation of the fragmenting device. Alternatively or additionally, aspiration channels adjacent a fragmenting device may clear the visual path of a visualization device (such as a camera of an endoscope with which the fragmenting device is delivered) to be able to substantially continuously view the fragmenting process without interference by the fragments, dust, etc., caused by such process because the proximity of the aspiration channel allows sufficiently rapid clearing of the vicinity of the fragmenting device and the visualization device.

As may be appreciated, because aspiration of materials from the bodily mass generally applies negative pressure (e.g., suction, a vacuum source, etc.) to the treatment site, if the bodily mass is in a body cavity, care must be taken so that the cavity does not collapse from the aspiration being performed. For instance, excessive suction (negative pressure aspiration) may cause injury such as collapse, bruising, and/or bleeding, and/or may obscure the field of view (e.g., of a visualization device used during use of the fragmenting device) if pressure within the anatomical cavity is not carefully monitored and balanced, such as by refilling the anatomical cavity concurrently with aspiration of materials therefrom. Alternatively or additionally, anatomical cavities, such as within a kidney, may be filled with a relatively low volume of fluid (e.g., to maintain desired distension of an organ such as the kidney) and may be inadvertently over-filled (over-pressurized) from excess fluid if sufficient care is not taken to balance pressure within the anatomical cavity. Moreover, a low volume of fluid and/or excess application of energy to pulverize the bodily mass (e.g., from lasing), can overheat the anatomical site, potentially burning surrounding tissue. Other complications may occur if the delicate pressure balance within an anatomical structure is not carefully maintained. For instance, in the case of removal of materials from a kidney with irrigation and aspiration, high renal pelvic pressure (e.g., intrarenal pressure, or IRP, such as due to high irrigation rate) may cause such complications as renal damage, fever, systemic inflammatory response syndrome (SIRS), sepsis, and other adverse conditions. The medical professional would have to wait until homeostasis within the anatomical cavity is restored before the aspiration and/or fragmentation can be resumed.

The principles of the present disclosure address the above concerns by providing in some embodiments a device and system with an outlet channel (e.g., for aspiration, suction, vacuum/negative pressure, etc.), a distal inlet channel (e.g., for irrigation, flushing of debris, etc.), and a proximal inlet channel (e.g., for irrigation), with the outlet channel being coupled to a drain valve which is configured to selectively provide passive or active drainage of material from the outlet channel. The distal inlet channel is generally positioned distal to the proximal inlet channel and thus closer to the distalmost end (e.g., the free, terminal end) of the system. More particularly, the proximal inlet channel may generally remain proximal to the fragmenting device and/or the aspirating device (e.g., the distal opening of the outlet channel) during use, whereas the distal inlet channel may remain distal to the proximal inlet channel (and optionally also distal to the outlet channel) during use.

In some aspects, the fragmenting device is a laser device which includes a laser fiber which may be powered by a laser console to supply sufficient energy to a distal end thereof to fragment or pulverize a bodily mass. It will be appreciated that terms such as fragment, pulverize, break up, etc., a bodily mass may be used interchangeably herein without intent to limit unless specified. In some embodiments, the laser is turned off by a signal from a pressure/temperature sensor operatively associated with the system, such as when the fluid level in the kidney is low. The distal inlet channel may supply/refill fluid to flush away and/or to suspend fragments of materials pulverized by the laser device and/or to provide cooling fluids to reduce elevated temperatures which may be created by the laser device. In some aspects, elevated temperatures and/or dust may be aspirated by the outlet channel, such as to clear the view of a visualization devices (e.g., of a medical scope).

In some aspects, the fragmenting device is a passive device (e.g., not independently steerable, although optionally steerable by another device, such as a medical scope through which the fragmenting device extends) which may be delivered to an anatomical site within a first tubular elongate member such as within a sheath. A steerable flexible elongate member may be operably associated with (e.g., inserted into) the first tubular elongate member to provide steerability to the fragmenting device. In some aspects, the fragmenting device is a laser fiber. Alternatively or additionally, the fragmenting device extends through a lumen through the steerable flexible elongate member, such as through a working channel of a medical scope. In some aspects, the fragmenting device may be utilized while still within (proximal to the distalmost end of) the first tubular elongate member. In some aspects, the fragmenting device is operated after being advanced distal to the distalmost end of the steerable flexible elongate member. In some aspects, the fragmenting device is advanceable distal to the distalmost end of the first tubular elongate member. In some aspects, both the fragmenting device and the steerable flexible elongate member are advanceable distal to the distalmost end of the first tubular elongate member, such as prior to operating/activating the fragmenting device.

In some aspects, a visualization device (steerable or passive) is used to visualize operation of the fragmenting device, and/or other procedures performed with the system of the present disclosure. In some aspects, the visualization device is positioned in the vicinity of the fragmenting device to be sufficiently close to provide informative visualization of the fragmenting operation being performed. In such instances, the distal inlet channel may remain adjacent such visualization device, such as to flush debris from the viewing path of the visualization device. For instance, in some embodiments, the fragmenting device is delivered through a lumen (e.g., working channel) of a tubular elongate member which also delivers a visualization element. The distal inlet channel may be formed through the tubular elongate member, and, in some cases, may be coextensive (i.e., through) the lumen through which the fragmenting device is delivered. As such, the distal inlet channel may remain closer than the proximal inlet channel to the fragmenting device as the fragmenting device is advanced into the anatomical site. In some embodiments, such as described above, the fragmenting device may be delivered through a working channel of a medical scope (e.g., endoscope, ureteroscope, flexible ureteroscope, laparoscope, etc.) having a visualization device such as a camera, laser optics, etc. Optionally, the distal inlet channel remains adjacent the visualization device, such as immediately adjacent, particularly if the working channel of a medical scope defines the distal inlet channel (and, thus, the opening to the distal inlet channel is adjacent the distal end of the medical scope) and a visualization element is at the distal end of the medical scope.

In accordance with various principles of the present disclosure, the above-described first tubular elongate member (through which may be delivered a fragmenting device of a system formed in accordance with various principles of the present disclosure) may be delivered through a second tubular elongate member. As such, the first tubular elongate member may alternately be referenced herein as an inner tubular elongate member or inner sheath, and the second tubular elongate member may alternately be referenced herein as an outer tubular elongate member or outer sheath. The outer sheath may surround the inner sheath, thereby protecting the anatomical passage (e.g., ureter walls) from the inner sheath (e.g., from impact or injury by the inner sheath) as the inner sheath is maneuvered/inserted/withdrawn with respect to the target site (e.g., inside the ureter and kidney). The space between the inner sheath and the outer sheath may define the proximal inlet channel in accordance with various principles of the present disclosure.

It will be appreciated that the outer sheath need not extend to the distalmost end of the inner sheath. In embodiments in which, as described above, a distal inlet channel is defined through the inner sheath, such as through a tubular elongate member extending therethrough, an opening to such inlet channel remains adjacent to the distalmost end of the inner sheath, thereby defining a distal inlet channel. In some aspects, the distalmost end of the proximal inlet channel remains distal to the distalmost end of the outlet channel. In some aspects, the outlet channel, defined between the outer sheath and the inner sheath, remains proximally spaced from the distalmost end of the inner sheath and thus proximal to the distal inlet channel. In some aspects, the proximal inlet channel is proximal and external to the distalmost end of the outlet channel. In some aspects, the distal inlet channel is the working channel of a medical scope. In some aspects, the distal inlet channel (e.g., defined by the medical scope) can be extended distally beyond the distal end of the outlet channel and/or retracted proximally into the outlet channel. In some aspects, the distal end of the medical scope may be retracted further back than the proximal inlet channel (e.g., to perform virtual basketing of a bodily mass). The proximal inlet channel provides sufficient irrigation fluid at such location to ensure proper operation of the system of the present disclosure (e.g., performing and meeting operational expectations thereof, such as by preventing overheating, collapse of an anatomical cavity, flushing debris, etc., such as described above). For instance, in embodiments in which a working channel of a medical scope defines a distal inlet channel, the distal end of the outer sheath (and thus the inlet opening to the proximal inlet channel defined between the outer sheath and the inner sheath) may remain proximal to the distalmost end of the inner sheath to allow the proximal inlet channel to meet operational expectations thereof.

In view of the above, a system formed in accordance with various principles of the present disclosure provides a fragmenting/pulverizing device with a distal or forward-facing aspiration channel with direct scope vision and two inlet channels. The proximal inlet channel can be used to replenish fluid at the treatment site without the velocity or fluid pressure of the inlet flow disrupting/scattering stone fragments. The distal inlet channel (e.g., defined through a scope air/water channel) may also be used to replenish fluid at the treatment site. Alternatively or additionally, the distal inlet channel may be used to locally flush the vicinity of a visualization device to remove air bubbles, clear the field of view, move/relocate stones and/or debris, and/or to clear the lumen within the inner sheath. The medical professional may elect when to use each inlet channel—either individually/separately, or simultaneously.

In some aspects, principles of the present disclosure may be applied to treat (lase, pulverize, fragment, dust, remove, destroy, reduce the size of, etc.) renal stones. Renal stones can be of different sizes, shapes, hardnesses, etc., and can be located anywhere in the kidney or in the renal system. Various configurations of devices and systems described herein, including various combinations and subcombinations of elements or components thereof, may be used in accordance with various principles of the present disclosure, such as, for example, to treat renal stones and/or other bodily masses. Not all embodiments will be needed or used for a given treatment protocol. It will be appreciated that fragmenting (e.g., lasing) of renal stones or other bodily masses may be performed within the inner sheath (e.g., when positioned distal to the outer sheath), while larger masses generally would need to be fragmented outside the inner sheath and into smaller particles before they can be aspirated into the inner sheath or through the outlet channel. For example, bodily masses which are too large to fit within the working channel of the inner sheath or the outlet channel may be suctioned against the distal end of the inner sheath and lased or otherwise fragmented such as in that position. In some aspects, smaller bodily masses may be suctioned into the inner sheath and fragmented therein. In some aspects, the distal end or edge of the inner sheath is used as an indicator in sizing the bodily mass which may fit into the inner sheath. In some aspects, the inner sheath may be formed of a laser-resistant type material along a distal portion or optionally along the entire length thereof.

It will be appreciated that a medical professional may use other/additional equipment, tools, devices, etc., to facilitate stone fragmentation and/or for other tasks. In some aspects, various tools may be inserted through the working channel of a medical scope and/or may be used as a biasing member that may be insertable through the outlet channel formed between the inner and outer sheath so as to bias the inner member off center in the outlet channel. Examples of additional tools which may be used include a retrieval basket or other tools to reposition a bodily mass. For instance, it may be desirable or medically indicated to move a renal stone from a lower pole to an upper pole of a kidney for easier access thereto. Alternatively or additionally, the medical professional may aspirate and/or irrigate during the fragmenting procedure (e.g., lasing) to clear the field of view and/or to suction out fragments and/or elevated temperatures concurrently and/or between fragmenting episodes and/or after fragmenting has been performed to aspirate out fragments of the bodily mass.

The various systems described herein are referenced as fragmenting and/or fragment-removing systems. However, the various devices, apparatuses, assemblies, systems, etc., of the present disclosure may be configured to either fragment, or remove, or both fragment and remove bodily masses or parts thereof; and/or aspirate other materials; and/or irrigate treatment sites; and/or sense or otherwise determine conditions at a treatment site; and/or perform various other operations related to fragmenting and/or removing fragments from a treatment site. It will be appreciated that reference may be made to terms such as irrigate, flush, etc. (including other grammatical forms thereof), interchangeably and without intent to limit. It will further be appreciated that reference may be made to terms such as aspirate, suction, remove, apply vacuum or vacuum or negative pressure, etc. (including other grammatical forms thereof), interchangeably and without intent to limit.

The present disclosure is not limited to only the embodiments specifically described herein. The following description is of illustrative examples of embodiments only, and is not intended as limiting the broader aspects of the present disclosure.

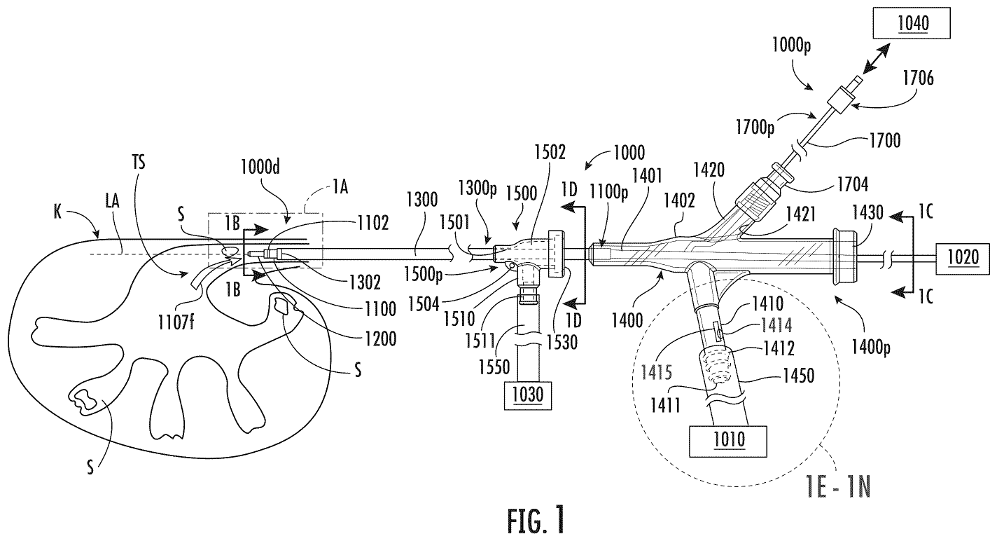

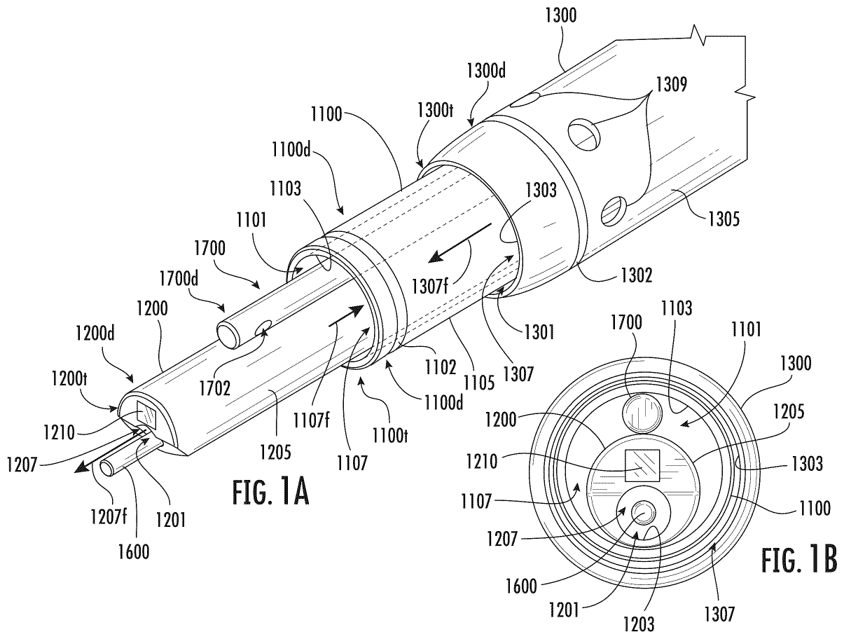

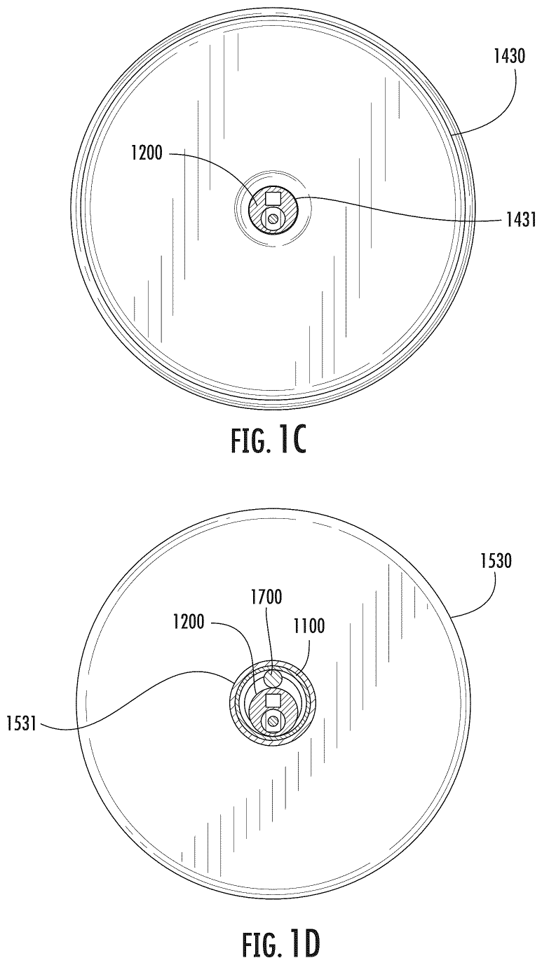

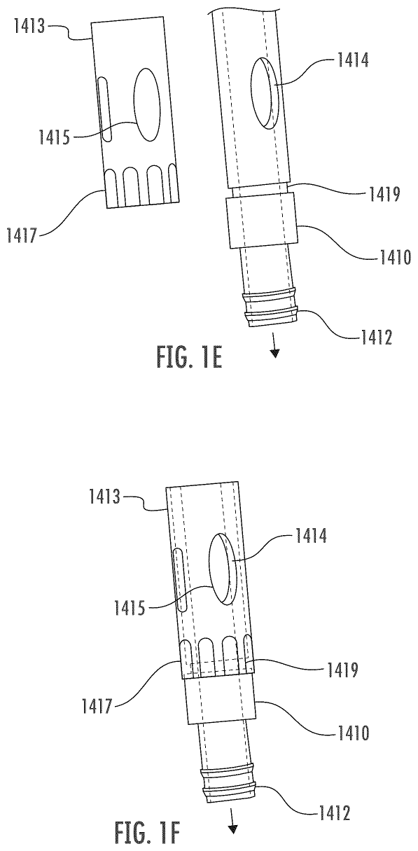

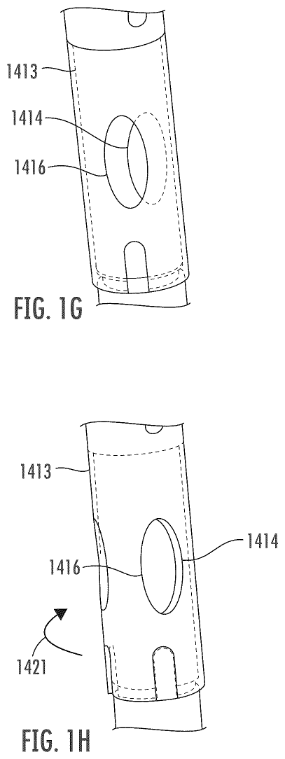

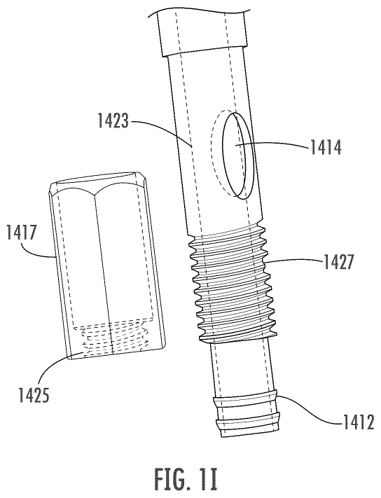

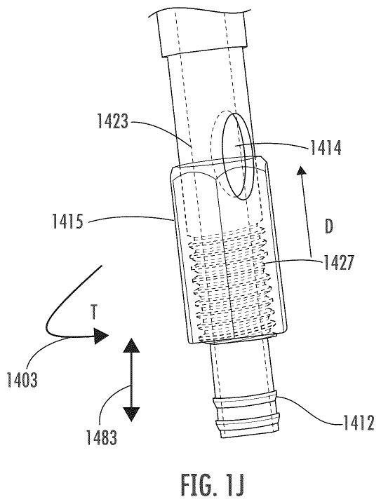

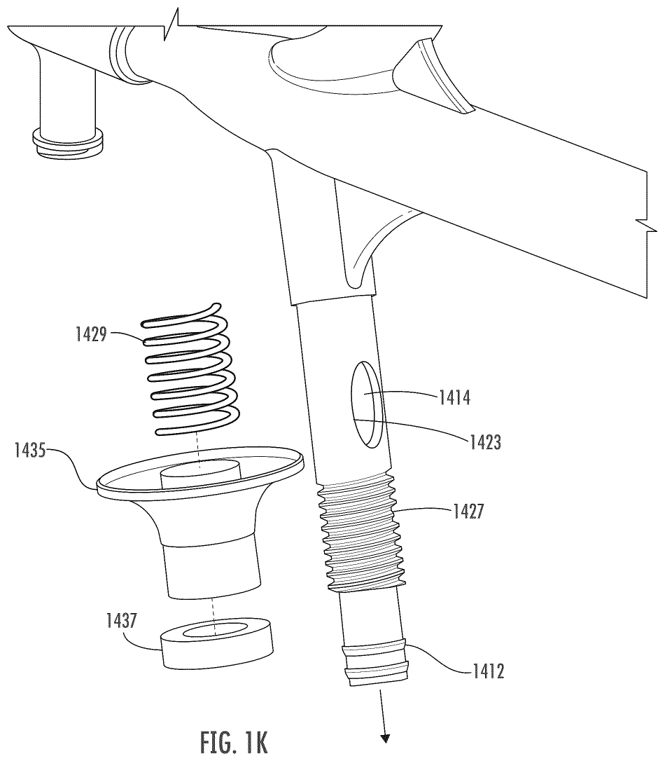

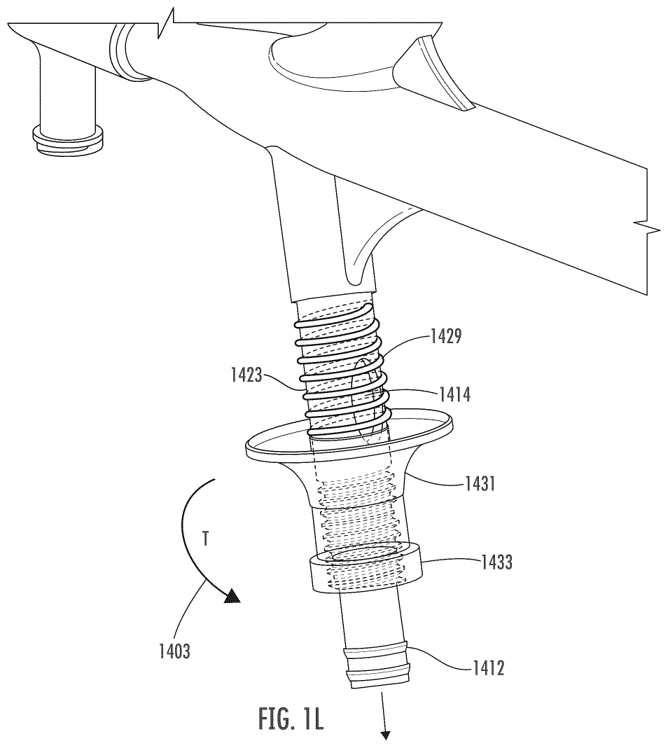

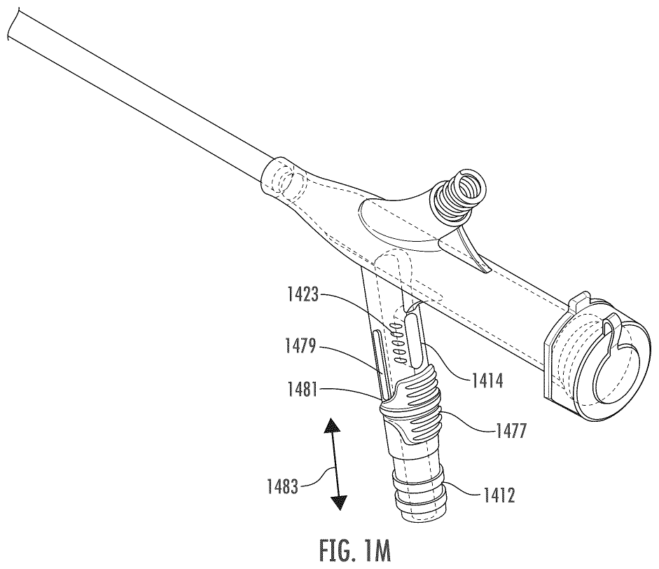

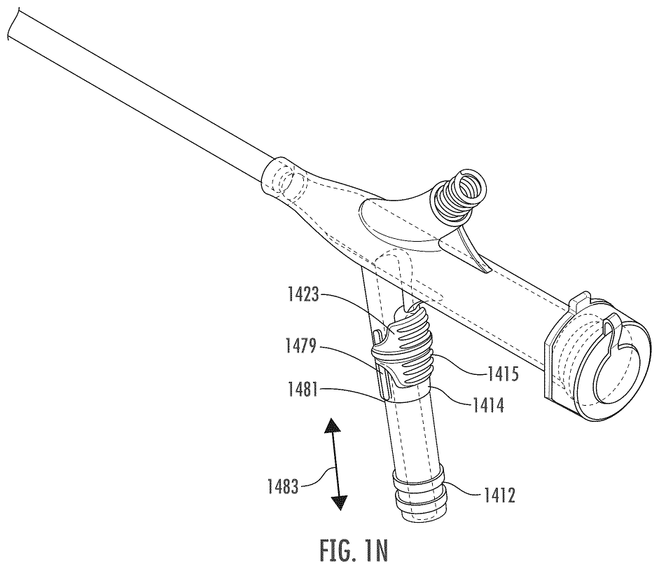

Turning now to the drawings, an example of an embodiment of a fragmenting and/or fragment-removing system 1000 formed in accordance with various principles of the present disclosure is illustrated in FIG. 1 with respect to a schematic example of an embodiment of a treatment site T. It will be appreciated that reference may be made herein to a treatment site, anatomical site, target site, etc., interchangeably and without intent to limit. Furthermore, it will be appreciated that reference made herein to an anatomical cavity as an example of an anatomical site, but the principles of the present disclosure need not be so limited.

The fragmenting and/or fragment-removing system 1000 illustrated in FIG. 1 includes an inner sheath 1100 defining the distal outlet channel 1107 (see, e.g., the end view of FIG. 1B, showing the distal outlet channel 1107 into which suction flow 1107f, shown in FIG. 1, is directed) of the fragmenting and/or fragment-removing system 1000 through which a bodily mass and/or fragments thereof may be removed from a treatment site. The fragmenting and/or fragment-removing system 1000 optionally includes a tubular elongate member 1200 extending within/through the inner sheath 1100. Additionally or alternatively, the fragmenting and/or fragment-removing system 1000 includes an outer sheath 1300 extending over inner sheath 1100 (i.e., through which the inner sheath 1100 extends). The distal end 1000d of the fragmenting and/or fragment-removing system 1000 (including distal ends of one or more of the inner sheath 1100, the tubular elongate member 1200, and/or the outer sheath 1300) is insertable into a patient's body (e.g., transluminally, such as through a naturally-occurring body orifice; percutaneously; or otherwise) to a treatment site T. A body 1400 (e.g., main body, connector, handle, etc.) may be provided along a generally proximal end 1000p of the fragmenting and/or fragment-removing system 1000 to couple (e.g., to provide fluid connections among) various components of the fragmenting and/or fragment-removing system 1000, as described in further detail below. Optionally, the proximal end 1100p of the inner sheath 1100 is operatively coupled with the body 1400. Optionally, the proximal end 1300p of the outer sheath 1300 is operatively coupled with a fitting 1500, which, in turn, may be operatively coupled with the inner sheath 1100, as described in further detail below.

As illustrated in further detail in FIG. 1A, showing detail area 1A in FIG. 1, the inner sheath 1100 defines a lumen 1101 (e.g., an inner lumen) therethrough. The lumen 1101 may be fluidly coupled at or along a proximal end 1100p of the inner sheath 1100 with a suction source 1010, as illustrated schematically in FIG. 1. For instance, the proximal end 1100p of the inner sheath 1100 may be fluidly coupled with a lumen 1401 (e.g., axial lumen) defined through the body 1400/housing 1402 of the body 1400, and, in turn, with a lumen 1411 defined through a first sidearm 1410 (e.g., a straight sidearm) of the body 1400. In some aspects, the inner sheath 1100 may be coupled with the body 1400. In some aspects, the first sidearm 1410 and the body 1400 are sealed to the atmosphere, even when additional instruments (e.g., a biasing member and/or a medical scope) are inserted therein. In some aspects, the lumen 1411 of the first sidearm 1410 extends transverse to the lumen 1101 defined through the inner sheath 1100.

The lumen 1411 of the first sidearm 1410, in turn, may be fluidly coupled with a suction connector tube 1450 (e.g., engaged with barbs 1412 on the first sidearm 1410). The suction connector tube 1450 may be coupled, in turn, with the suction source 1010. The suction source 1010 may have any form or configuration such as known to those of ordinary skill in the art, and thus is schematically illustrated with any particular design, the present disclosure not being limited in this regard. It will be appreciated that terms such as suction, aspiration, outlet, vacuum, vacuum pressure, removal, evacuation, etc., including various grammatical forms thereof, may all be used to refer to the withdrawal of the fragments, particles, dust, etc., of the bodily mass to be removed from the anatomical site. As fluidly coupled with a suction source 1010, the lumen 1101 of the inner sheath 1100 may be considered to define the distal outlet channel 1107 of the fragmenting and/or fragment-removing system 1000. That is, an outlet channel such as the distal outlet channel 1107 can be manifested as at least a portion of a lumen such as the lumen 1101.

The inner sheath 1100 and/or the outer sheath 1300 and/or the body 1400 and associated fittings may be formed of a transparent or clear material, such as to allow viewing of materials aspirated therethrough, or an opaque material, the present disclosure not being limited in this regard. In some aspects, the inner sheath 1100 may have more than one section. For instance, in some embodiments, the inner sheath 1100 is made of at least of a distal section and a proximal section. The material of the distal section may be selected to be laser resistant, such as to withstand damage from a fragmenting device such as a laser which may be extended into and used within the inner sheath 1100 (as described in further detail below). For example, the distal section may be made of Expanded Polytetrafluoroethylene (EPTFE) or lined with EPTFE. Additionally or alternatively, the material of the distal section of the inner sheath 1100 may be soft and/or flexible, have high temperature stability (e.g., a melting point of at least about 380° C.), and/or made in different densities/column strengths. Optionally, the material of the distal section of the inner sheath 1100 may be reinforced, such as with an EPTFE coil. A radiopaque marker 1102 may be provided along the distal end 1100d of the inner sheath 1100, such as to facilitate visualization thereof (e.g., with fluoroscopy), such as during navigation, placement, use, etc., as described in further detail below. The proximal section of the inner sheath 1100 is operatively associated with the body 1400, thereby coupling the lumen 1101 defined through the inner sheath 1100 with the first sidearm 1410 of the body 1400, as described above. The lumen 1101 of the inner sheath 1100 may also be fluidly coupled with a second sidearm 1420 of the body 1400 which may be used for insertion of further devices through the lumen 1101, such as described in further detail below.

As illustrated in the detail view in FIG. 1A of the detail area 1A in FIG. 1, the optional tubular elongate member 1200 may extend (e.g., axially, longitudinally, etc.) through the lumen 1101 of the inner sheath 1100. With such arrangement, the above-described distal outlet channel 1107 is defined between the inner surface 1103 of the inner sheath 1100 (e.g., defining the inner diameter of the inner sheath 1100) and an outer surface 1205 of the tubular elongate member 1200 (e.g., defining the outer diameter of the tubular elongate member 1200). The tubular elongate member 1200 may be inserted into the lumen 1101 of the inner sheath 1100 via the proximal end 1400p of the body 1400. More particularly, a seal 1430 may be operatively associated with the proximal end 1400p of the body 1400 and may include at least one through hole 1431 through which the tubular elongate member 1200 is insertable into the lumen 1101 of the inner sheath 1100. The through hole 1431 (see, e.g., the end view along line 1C-1C of FIG. 1, as illustrated in FIG. 1C) may be sized, shaped, configured, and/or dimensioned to sealingly engaging the outer surface 1205 of the tubular elongate member 1200 to inhibit/prevent leakage. For example, the through hole 1431 may have a diameter slightly smaller than the outer diameter of the tubular elongate member 1200 such that when the tubular elongate member 1200 is inserted into the through hole 1431, the seal 1430 seals the distal outlet channel 1107 formed between the inner sheath 1100 and the tubular elongate member 1200. In some aspects, the seal 1430 is assembled, glued, tethered, or otherwise coupled with respect to the body 1400 such that it will not be lost, or needs be assembled (or misassembled) by the user. The seal may be screw threaded, friction fitted, or otherwise removably mounted on the proximal end 1400p of the body 1400, or fixedly mounted thereon (e.g., welded), the present disclosure not being limited in this regard. In some embodiments the seal is a Tuohy Borst adaptor.

As illustrated in FIG. 1A, and in the end view of FIG. 1 illustrated in FIG. 1B, the tubular elongate member 1200 defines a lumen 1201 therethrough. The lumen 1201 may be fluidly coupled with a fluid source 1020, such as at a proximal end thereof. Although the proximal end of the tubular elongate member 1200 is not illustrated, the configuration thereof may be readily understood by those of ordinary skill in the art, and thus does not require illustration to facilitate understanding thereof. The fluid source 1020 provides fluid (e.g., saline, etc.) for irrigation, flushing, etc., of the treatment site, such as known to those of ordinary skill in the art, and thus is schematically illustrated, the present disclosure not being limited in this regard. As fluidly coupled with a fluid source 1020, the lumen 1201 defines a first inlet channel 1207 of the fragmenting and/or fragment-removing system 1000. In some aspects, the distalmost end 1200t of the tubular elongate member 1200 is translatable between a proximal position, within the inner sheath 1100 and proximal to the distalmost end 1100t of the inner sheath 1100, and a distal position extending outside the inner sheath 1100 and distal to the distalmost end 1100t of the inner sheath 1100, such as to modify the position at which the first inlet channel 1207 supplies fluid into the treatment site T, as discussed in further detail below. In some aspects, the tubular elongate member 1200 may define a lumen defining a fluid inlet coupled to a fluid source that is separate from the lumen through which the fragmenting device 1600 extends.

In the illustrated example of an embodiment, the tubular elongate member 1200 is a medical scope. As may be appreciated by those of ordinary skill in the art, medical scopes have various components providing functionalities as known in the art for transluminal delivery to a treatment site within a patient's body. Examples of medical scopes include, without limitation, flexible ureteroscopes, arthroscopes, bronchoscopes, colonoscopes, cystoscopes, duodenoscopes, gastroscopes, hysteroscopes, laparoscopes, and ureteroscopes. In view of the example of an embodiment of a treatment site being a kidney, the illustrated example of an embodiment may be a flexible ureteroscope which may include a steering system (such as with pull wires manipulable to deflect the distal end of the scope shaft as needed during a procedure). However, a fragmenting and/or fragment-removing system 1000 formed in accordance with various principles of the present disclosure may include and/or use other types of medical scopes, the present disclosure not being limited in this regard. In the example of an embodiment illustrated in FIG. 1, FIG. 1A, and FIG. 1B, the lumen 1201 defining the first inlet channel 1207 of the tubular elongate member 1200 of the fragmenting and/or fragment-removing system 1000 is a working channel 1203 of a medical scope such as a ureteroscope. The working channel 1203 may be used for irrigation of the treatment site T (e.g., during lasing with the fragmenting device 1600), and/or to flush, clean, move, group, and/or disperse dust/fragments.

As further illustrated in the detail view in FIG. 1A, the optional outer sheath 1300 defines a lumen 1301 (e.g., an outer lumen) extending therethrough and through which the inner sheath 1100 extends. In accordance with various principles of the present disclosure, the outer sheath 1300 may be fluidly coupled with a fluid source 1030. For instance, the proximal end 1300p of the outer sheath 1300 may be fluidly coupled with a fitting 1500, and, in turn, with a lumen 1511 port defined through a sidearm 1510 of the fitting 1500. The sidearm 1510, in turn, may be fluidly coupled with a suction source 1030, such as via a connection tube 1550. Fluid from the fluid source 1030 may flow through the space between the inner sheath 1100 (e.g., the outer surface 1105 of the inner sheath 1100, such as defining an outer diameter of the inner sheath 1100) and the outer sheath 1300 (e.g., the inner surface 1303 of the outer sheath 1300, such as defining the inner diameter of the outer sheath 1300), such space thereby defining a proximal inlet channel 1307 of the fragmenting and/or fragment-removing system 1000. Fluid from the fluid source 1030 can flow from the proximal inlet channel 1307 into the treatment site T (as an inlet of fluid) distally from the distalmost end 1300t of the outer sheath 1300 (via the space between the outer surface 1105 of the inner sheath 1100 and the inner surface 1303 of the outer sheath 1300 just proximal to the distalmost end 1300t) and/or via distal holes/apertures 1309 formed along a distal end 1300d of the outer sheath 1300 just proximal to the distalmost end 1300t of the outer sheath 1300. The fluid source 1030 may be the same as or different from the fluid source 1020 to which the tubular elongate member 1200 is fluidly coupled. In some aspects, fluid supply to the first inlet channel 1207 and to the proximal inlet channel 1307 may be separately and independently controllable, Preferably, if the tubular elongate member 1200 and the outer sheath 1300 are coupled with same fluid source, fluid supply to each may be separately and independently controlled so that the fluid flow through the first inlet channel 1207 (defined through the tubular elongate member 1200) may be different from the fluid flow through the proximal inlet channel 1307 (defined between the outer sheath 1300 and the inner sheath 1100), the benefits of such arrangement being appreciated with reference to the further descriptions thereof provided below. Because the proximal inlet channel 1307 is defined between the outer sheath 1300 and the inner sheath 1100 (and thus proximal to the distalmost end 1100t of the inner sheath 1100), the proximal inlet channel 1307 generally remains proximal to the first inlet channel 1207. Accordingly, the proximal inlet channel 1307 may be considered and thus alternately referenced herein as a proximal inlet channel 1307, and the first inlet channel 1207 may be considered and thus alternately referenced herein as a distal inlet channel 1207.

It will be appreciated that in some embodiments, instead of defining an inlet channel, the outer sheath 1300 may define a secondary outlet channel between the inner surface 1303 thereof and the outer surface 1105 of the inner sheath 1100. In some aspects, the outer sheath 1300 may include a one or more second fluid inlet channels (not illustrated) extending longitudinally through a portion of the outer sheath 1300. The one or more second fluid inlet channel may be fluidly coupled to a same or different fluid source as the first inlet channel 1207. The presence of the second fluid inlet channel may permit an increased irrigation rate which may in turn improve (e.g., increase) drainage and removal of a bodily mass or fragments thereof from a body cavity, may improve (e.g., increase) visibility at a distal end of a medical device such as a ureteroscope, may improve (e.g., lower) temperature in a body cavity, and/or may improve (e.g. lower) pressure in a body cavity. In some aspects, the outlet channel is not fluidly coupled with negative pressure and may be considered a passive drain. In some aspects, the outlet channel is fluidly coupled with a suction source and therefore may be considered an active drain. In some aspects, an outer sheath 1300 is not used, and a system may simply have a single lumen (e.g., with a single inlet and a single outlet).

The outer sheath 1300 may be formed from a kink-resistant material. For instance, the distal end 1300d of the outer sheath 1300 may be more flexible than the proximal end 1300p of the outer sheath 1300, such as to facilitate insertion into the renal pelvis and/or to facilitate navigation thereof within a patient's body, such as to facilitate navigation of the inner sheath 1100 and/or the tubular elongate member 1200 which extend therethrough. In some aspects, at least a portion of both the distal end 1300d and the proximal end 1300p of the outer sheath 1300 may be reinforced, such as with a coil and/or braid. A radiopaque marker 1302 (such as illustrated in FIG. 1 and FIG. 1A) may be provided along the distal end 1300d of the outer sheath 1300, such as to facilitate visualization thereof (e.g., with fluoroscopy), such as during navigation, placement, use, etc., and/or to view the position of the distal end 1300d of the outer sheath 1300 with respect to the distal end 1100d of the inner sheath 1100. In some aspects, the outer sheath 1300 has a hydrophilic and/or friction-reducing coating over the outer surface 1305 thereof. A hydrophilic coating and/or friction-reducing coating on the outer sheath 1300 may facilitate insertion and advancement of the outer sheath 1300 into the patient. The use of a hydrophilic and/or friction-reducing coating may minimize discomfort and/or otherwise improve patient outcomes. These specialized sheaths offer numerous advantages over traditional sheaths, including improved patient comfort, reduced risk of infection, and faster recovery times.

As described above, the proximal end 1300p of the outer sheath 1300 may be connected to a sidearm 1510 of a fitting 1500. The lumen 1511 defined through the sidearm 1510 may extend transverse to the lumen 1301 through the outer sheath 1300 and the lumen 1501 through the housing 1502 of the fitting 1500. The sidearm 1510 may extend such that the lumen 1511 therethrough is substantially perpendicular to the lumen 1501 through the fitting housing 1502 (such as in the example of an embodiment illustrated in FIG. 1), or may be at angle greater or less than 90° with respect to the outer sheath 1300. In some embodiments, a seal 1530 is provided at a proximal end 1500p of the fitting 1500 and fitted over the outer surface 1105 of the inner sheath 1100. The seal 1530 may have a through hole 1531 (such as illustrated in FIG. 1D) that is slightly smaller than the outer diameter of the outer surface 1105 of the inner sheath 1100, such as to seal the proximal inlet channel 1307 defined between the outer sheath 1300 and the inner sheath 1100. In some aspects, the seal 1530 may be attached to the outer sheath 1300, such as to prevent loss and/or to save an assembly step for the user. In some aspects, the outer sheath 1300 is advanceable further into the renal pelvis. Coupling of the seal 1530 to the housing 1502 may allow the advanceability of the outer sheath 1300. In some aspects, a suture hole 1504 is defined in the housing 1502 of the fitting 1500 configured for securing of a suture to the outer sheath 1300 and to the patient in a manner known those of ordinary skill in the art.

In accordance with various principles of the present disclosure, the inlet flow through the distal inlet channel 1207 counteracts the suction/aspiration flow through the distal outlet channel 1107, such as when the distalmost end 1200t of the tubular elongate member 1200 is retracted into the inner sheath 1200 and proximal to the distalmost end 1100t of the inner sheath 1100. One manner of avoiding or at least minimizing such counteraction is to lower or turn off the irrigation supplied by the distal inlet channel 1207, thereby allowing the suction through the distal outlet channel 1107 to dominate. In such instance, the replenishing fluid (fluid maintaining a desired homeostatic pressure at the treatment site T) delivered into the treatment site T is mainly delivered by the proximal inlet channel 1307.

In accordance with various principles of the present disclosure, as described above, and as may be appreciated, such as with reference to FIG. 1A and FIG. 1B, the distal outlet channel 1107 of the fragmenting and/or fragment-removing system 1000 illustrated in FIG. 1 (through which fragments are removed from a treatment site T) may be considered to be defined by (e.g., by the space between) the inner diameter of the inner sheath 1100 and the outer diameter of the tubular elongate member 1200. Further in accordance with various principles of the present disclosure, it may be desirable to maximize the size/area (e.g., generally in a direction transverse to the direction of flow through the distal outlet channel 1107, such as transverse to a longitudinal axis LA of the fragmenting and/or fragment-removing system 1000) of the distal outlet channel 1107. In order to maintain a desired spacing between the inner surface 1103 of the inner sheath 1100 and the outer surface 1205 of the tubular elongate member 1200, a biasing member 1700 may be positioned within the inner sheath 1100, such as illustrated in FIG. 1A and FIG. 1B, to maintain a space between the inner surface 1103 of the inner sheath 1100 and the outer surface 1205 of the tubular elongate member 1200. For instance, as illustrated in FIG. 1B, the biasing member 1700 may occupy one side of the lumen 1101 defined within the inner sheath 1100, while the tubular elongate member 1200 occupies the other side of the lumen 1101. In another words, the space between the inner surface 1103 of the inner sheath 1100 and the outer surface 1205 of the tubular elongate member 1200 (which may be referenced herein as a gap therebetween) is shifted or maximized by the presence of the biasing member 1700, positioning the tubular elongate member 1200 on the opposite side of the lumen 1101 than the location of the biasing member 1700 to allow larger fragments to pass out the distal outlet channel 1107 defined along the length of the inner sheath 1100 alongside the length of the biasing member 1700. In some aspects, the biasing member 1700 biases the tubular elongate member 1200 against a first area along the inner surface of the inner sheath 1100 to maximize a distance between a second area along the inner surface of the inner sheath 1100 opposite the first area to maximize a dimension of the distal outlet channel 1107 transverse to direction of fluid flow along the longitudinal axis LA of the fragmenting and/or fragment-removing system 1000. In some aspects, as illustrated in FIG. 1B, the biasing member 1700 has a generally circular profile, however, other shapes or configurations are within the scope of the present disclosure. In some aspects, one or more biasing members are inserted into the inner sheath 1100.

In some aspects, the biasing member 1700 is an elongate member, such as a flexible elongate member. In some aspects, the biasing member 1700 is a monofilament extrusion with a flexible distal end or a guidewire with a flexible distal end or the like. In some aspects, the biasing member is a microcatheter, a catheter, a balloon catheter, a retrieval device, an ultraviolet light (e.g., black light/UV-A light), a biopsy device, and/or or a medical elongated device. In some aspects, the biasing member 1700 has a flexible distal end 1700d. In some aspects, the biasing member 1700 may be a steerable elongate member. In some aspects, the steerable elongate member is configured with a visualization device (e.g., imager, such as a camera chip). In some aspects, the inner sheath 1100 is a passive element (i.e., not capable of actively being steered), and a steerable biasing member 1700 thus allows steering or other control and/or navigation of the inner sheath 1100 or at least the distal end 1100d thereof. The distal end 1700d of the biasing member 1700 may flex independently of the inner sheath 1100 (even if such flexion then causes the inner sheath 1100 to flex as well), as the biasing member 1700 need not be attached to the inner sheath 1100. In some aspects, the biasing member 1700 is formed of or at least covered with EPTFE (and/or another laser-resistant material, such as for safety).

In some aspects, a connector 1704, such as a Tuohy Borst adaptor, can be releasable coupled to the second sidearm 1420 of the body 1400 and/or the biasing member 1700 (e.g., to the exterior thereof), such as illustrated in FIG. 1, to allow adjustment of the length of the biasing member 1700 relative to the length of the inner sheath 1100. For instance, in the example of an embodiment illustrated in FIG. 1, the biasing member 1700 may be inserted into the fragmenting and/or fragment-removing system 1000, such as into the inner sheath 1100 thereof, with the assistance of the body 1400. More particularly, the biasing member 1700 may be inserted into a sidearm of the body 1400, such as the second sidearm 1420 (e.g., a curved sidearm 1420).

The second sidearm 1420 has a lumen 1421 defined therethrough in fluid communication with the lumen 1401 defined through the body 1400/housing 1402 of the body 1400 and thus in fluid communication with the lumen 1101 defined through the inner sheath 1100. Insertion of the biasing member 1700 into the second sidearm 1420 of the body 1400 thus facilitates insertion of the biasing member 1700 into the inner sheath 1100 which extends into the body 1400, as described above. In some aspects, the biasing member 1700 may be extended distal to the distalmost end 1100t of the inner sheath 1100. However, when aspirating larger masses (e.g., masses closer to or even larger than the largest dimension of the distal outlet channel 1107), the biasing member 1700, and optionally also the tubular elongate member 1200, can be withdrawn slightly into the inner sheath 1100 (proximal to the distalmost end 1100t of the inner sheath 1100) to allow the entire distal outlet channel 1107 and/or the entire lumen 1101 of the inner sheath 1100 to accommodate a larger mass therein, such as described in further detail below.

In some aspects, the biasing member 1700 incorporates a sensor element capable of sensing and/or indicating one or more conditions at the treatment site T. For instance, the biasing member 1700 may include a temperature and/or pressure sensor, such as at or along a distal end 1700d thereof. Alternatively or additionally, the sensor element includes a force sensor, an optical pressure sensor, a magnetic sensor, a color senor, a light sensor, a distance sensor, a time-of-flight sensor, and/or a biological-material sensing material (e.g., capable of sensing antibodies, enzymes, cell receptors which interacts with analyte(s), etc.). Other types of sensors may determine the size and composition of the bodily mass (e.g., renal stone) being targeted. The sensor element may include an optical, physicochemical, and/or piezoelectric transducer that translates the biological signal to electrical and/or electric signals. In some aspects, the biasing member 1700 with a sensor element may be mounted on (e.g., glued to) the outer surface of the tubular elongate member 1200.

FIG. 1E illustrates a detail view of detail area 1E-1N in FIG. 1. As illustrated in FIG. 1E, the first sidearm 1410 can include a vent 1414, a coupling mechanism such as a groove 1419 to couple with a corresponding coupling mechanism such as hooks 1409 of a cover 1413, and a coupling mechanism such as barbs 1412 to couple with a suction source. For instance, as illustrated in FIG. 1F, the hooks 1409 can be coupled to the groove 1419.

The cover 1413 can be configured to selectively cover the vent 1414. For instance, the cover 1413 can be moved relative to the vent 1414 to selectively expose or cover the vent 1414. In some embodiments, the cover 1413 can be continuous (e.g., without an aperture) and may be moved relative to the vent 1414 to selectively expose or cover the vent 1414. In some embodiments, the cover 1413 is a movable cover that is disposed on an outer surface of the first sidearm 1410. As detailed herein, in such embodiments, the cover 1413 is configured to expose the vent 1414 when the cover 1413 is in the first position and is configured to cover (e.g., block) at least a portion of the vent 1414 when the cover 1413 is in the second position.

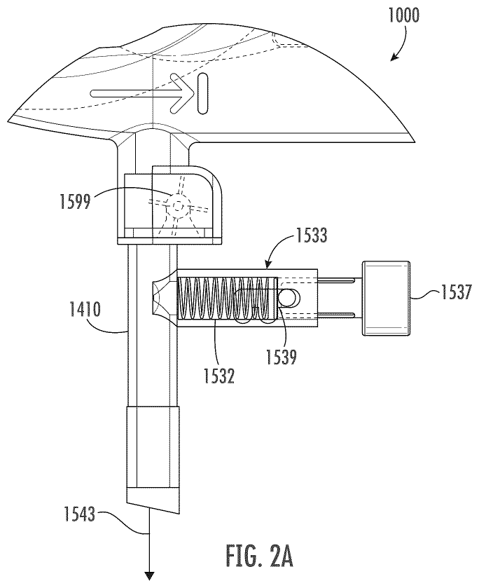

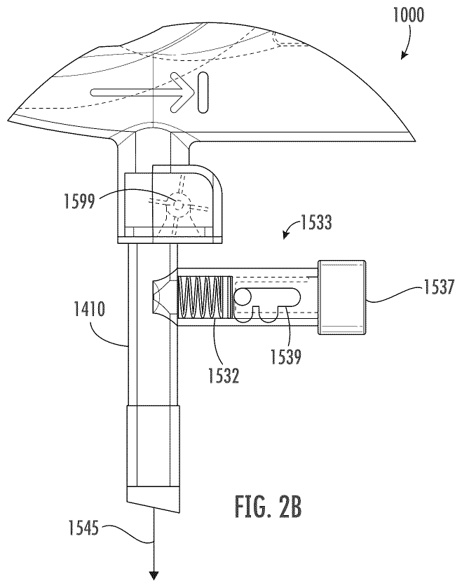

That is, vent 1414 and the cover 1413 may together form a drain valve when employed together, for instance, in the configurations illustrated in FIGS. 1E-1N. Stated differently, in some embodiments, the drain valve includes a vent such as the vent 1414. For instance, alteration of a position of the cover 1413 relative to the vent 1414 can alter a flow of material through the first sidearm 1410 and/or alter a flow of material through the outlet channel. The cover can include an aperture 1415 that can be moved (e.g., translated, rotated, etc.) relative to the vent 1414 to reduce or increase air flow through the vent 1414 and thereby increase or reduce, respectively, an amount of suction imparted on any material in a lumen of the first sidearm 1410 and/or any material in the outlet channel. For example, when the vent 1414 is exposed (e.g., is partially exposed or is not covered by a solid surface of the cover 1413), material may be passively drained via the first sidearm 1410 and the outlet channel into a drain conduit that is coupled to the first sidearm 1410. Stated differently, when a drain valve formed at least in part by the vent 1414 and the cover 1413 is in a first position at which the vent 1414 is exposed (e.g., is not covered by a solid surface of the cover 1413), material may be passively drained via the first sidearm 1410 and the outlet channel into the drain conduit (e.g., as the passively drained material 1543 that is passively drained via the drain conduit 1541, as illustrated in FIG. 2A). That is, the material 1543 can be passively drained in the absence of any activation or action from a user (e.g., in the absence of moving a position of the cover 1413 and/or depressing a button). Such passive draining in the absence of any activation or action from a user may permit operation of a medical device at an increased irrigation rate which may in turn improve (e.g., increase) drainage and removal of a bodily mass or fragments thereof from a body cavity, may improve (e.g., increase) visibility at a distal end of a medical device such as a ureteroscope, may improve (e.g., lower) temperature in a body cavity, and/or may improve (e.g. lower) pressure in a body cavity.

Yet, the approaches herein provide for the selective activation of active drainage without any changing or removing equipment of a medical device. For instance, when the vent 1414 is at least partially (e.g., is entirely covered by a solid surface of the cover 1413), material may be actively drained via the first sidearm 1410 and the outlet channel into a drain conduit that is coupled to the first sidearm 1410. Stated differently, when the drain valve formed at least in part by the vent 1414 and the cover 1413 is in a second position at which the vent 1414 is covered (e.g., is entirely covered by a solid surface of the cover 1413), material may be actively drained via the first sidearm 1410 and the outlet channel into the drain conduit (e.g., as the actively drained material 1545 that is actively drained via the drain conduit 1541, as illustrated in FIG. 2B). That is, a suction source may be coupled to the drain conduit 1541 such that variation of the position of the cover 1413 relative to the vent 1414 can permit material to be either passively or actively drained via the drain conduit, as illustrated in FIG. 2A and FIG. 2B, respectively. The vent 1414 can extend from the lumen in the first sidearm to the environment (e.g., atmosphere) surrounding the first sidearm 1410. The first sidearm 1410 can include barbs 1412, which may be fluidly coupled with a suction connector tube such as the suction connector tube 1450. For instance, the barbs 1412 can engage with a first end of a drain conduit 1541, as illustrated in FIG. 2A, and a second end or another portion of the drain conduit 1541 can be engaged with the suction connector tube 1450.

While the vent 1414 and the aperture 1415 are both illustrated as being the same size and shape (e.g., oval) in FIG. 1E, in some embodiments the vent 1414 and/or the aperture 1415 can be different sizes and/or different shapes.

FIG. 1G illustrates a perspective view of an example of the detail area 1E-1N in FIG. 1. As illustrated in FIG. 1G, a cover 1413 can be an arcuate cover. The cover 1413 is in a first position where the vent 1414 is partially exposed (e.g., is partially aligned with a collet blank 1416 in the cover 1413), as illustrated in FIG. 1G. That is in some embodiments, the vent 1415 may be replaced with a solid surface such as the collet blank 1416. As mentioned, when in the first position such as illustrated in FIG. 1G, material may passively drain from the outlet channel to the drain conduit.

FIG. 1H illustrates a perspective view of an example of the detail area 1E-1N in FIG. 1. As illustrated in FIG. 1H, the cover 1413 is in a second position where the vent 1414 is covered (e.g., is overlaid and obstructed by the collet blank 1416 in the cover 1413). Namely, FIG. 1H illustrates the cover 1413 in a second position after the cover 1413 has been rotated in a direction 1403 about the vent 1414 in the first sidearm 1410 from the first position illustrated in FIG. 1G to the second position illustrated in FIG. 1H. As mentioned, when in the second position such as illustrated in FIG. 1G, material may actively drain from the outlet channel to the drain conduit.

FIG. 1I illustrates a perspective view of an example of the detail area 1E-1N in FIG. 1. FIG. 1I is similar to FIG. 1E, with the change that the arcuate cover 1413 has been replaced with an elongate nut 1417 including threads 1425 and the first sidearm 1410 includes threads 1427 and visual indicators of a state of the elongate nut 1417. The visual indicators 1423 can include marks, numerical, alphabetical, and/or other types of indicators suitable to indicate a position of the elongate nut 1417 (or other type of cover) relative to the vent 1414.

FIG. 1J illustrates a perspective view of an example of the detail area 1E-1N in FIG. 1. As illustrated in FIG. 1I, the cover in the form of the elongate nut 1417 can be in a first position where the vent 1414 is partially exposed (e.g., the nut is partially covering the vent 1414). As mentioned, when the cover such as the elongate nut 1417 is in the first position such as illustrated in FIG. 1J, material may passively drain from the outlet channel to the drain conduit. The elongate nut 1417 can be rotated (e.g., in direction 1403) and thereby can be moved in the direction 1483. For instance, the elongate nut 1417 can be moved to a second position (not shown) where the vent 1414 is covered (e.g., is overlaid by an inner surface of the elongate nut 1417) such that material may actively drain from the outlet channel to the drain conduit.

The elongate nut 1417 can have a length (in the direction 1483) that is equal to or greater than a length of the vent 1414 (in the direction 1483), as illustrated in FIG. 1J. The elongate nut 1417 (or other type of cover such as those described herein) having a length that is equal to or greater than a length of the vent 1414 can promote aspects herein such as selectively covering or exposing the vent 1414 to provide active drainage or passive drainage, as detailed herein.

FIG. 1K illustrates a perspective view of an example of the detail area 1E-1N in FIG. 1. FIG. 1K is similar to FIG. 1I, with the change that the elongate nut 1417 has been replaced with a combination of spring 1429, a knob 1435, and a nut 1437. The spring 1429, a knob 1435, and a nut 1437 can each disposed around a circumference of the first sidearm 1410. For instance, as illustrated in FIG. 1L, the spring 1429, the knob 1435, and the nut 1437 can be disposed around a circumference of the first sidearm 1410 and the spring 1429 can disposition the knob 1435 to contact the nut 1437. As illustrated in FIG. 1L, the knob 1435 can be in a first position where the vent 1414 is partially exposed. As mentioned, when in the first position such as illustrated in FIG. 1L, material may passively drain from the outlet channel to the drain conduit. The nut 1437 is configured to translate about the first sidearm 1410 by way of rotation of the nut 1437 along the threads 1427 of the first sidearm 1410. For instance, the nut 1437 can be rotated to cause the nut 1437 to translate toward the vent 1414 until the knob 1435 is in a second position at which the knob 1435 covers the vent 1414 (not illustrated). As mentioned, when in the second position, material may actively drain from the outlet channel to the drain conduit.

FIG. 1M illustrates a perspective view of an example of the detail area 1E-1N in FIG. 1. FIG. 1M is similar to FIG. 1I, with the change that the elongate nut 1417 has been replaced with a snap-on cover 1477. While FIG. 1M and FIG. 1N illustrate the presence of a seal (e.g., seal 1430 as illustrated in FIG. 1) in the form of a removable plug, the seal may be manifested as a different type of seal such as a self-sealing seal, etc. The seal may be utilized in various instances, for example to permit use of the fragmenting and/or fragment-removing system in the absence of a scope, etc. The snap-on cover 1477 can be an arcuate cover that includes tabs 1481 that are configured to friction fit or snap into corresponding slots 1479 on the outer surface of the first sidearm 1410, as illustrated in FIG. 1M. As illustrated in FIG. 1M, the snap-on cover 1477 is in a first position where the vent 1414 is partially exposed. As mentioned, when in the first position such as illustrated in FIG. 1M, material may passively drain from the outlet channel to the drain conduit.

FIG. 1N illustrates a perspective view of an example of the detail area 1E-1N in FIG. 1. As illustrated in FIG. 1H, the snap-on cover 1477 is in a second position where the vent 1414 is covered. Namely, FIG. 1N illustrates the snap-on cover 1477 in a second position after the snap-on cover 1477 has translated in the direction 1483 along the first sidearm 1410 from the first position illustrated in FIG. 1M to the second position illustrated in FIG. 1N. As mentioned, when in the second position such as illustrated in FIG. 1N, material may actively drain from the outlet channel to the drain conduit.

FIG. 2A illustrates an elevational view of an alternate arrangement of elements of a fragmenting and/or fragment-removing system with a drain valve in a first position in accordance with aspects of the present disclosure. FIG. 2B illustrates an elevational view of an alternate arrangement of elements of a fragmenting and/or fragment-removing system with a drain valve in a second position in accordance with aspects of the present disclosure. As illustrated in FIG. 2A and FIG. 2B, an example of an embodiment of a fragmenting and/or fragment-removing system 1000 may be substantially the same as the fragmenting and/or fragment-removing system 1000 illustrated in FIG. 1, other than the cover 1413 and the vent 1414 have been replaced with a drain valve 1533 including a visual flow indicator 1529. The drain valve 1533 may be coupled to a sidearm extending from a body of a medical device such as the fragmenting and/or fragment-removing system 1000. For instance, the drain valve 1533 may extend from the first sidearm 1410. The drain valve 1533 may be integral with (e.g., formed as a portion of) the sidearm or may be removably coupled to the sidearm. For instance, the drain valve may be removably coupled to the sidearm via a snap fitting, a screw/thread combination, or may otherwise be removably coupled to the sidearm. In some embodiments, drain valve 1533 may be coupled to a straight sidearm of the fragmenting and/or fragment-removing system 1000. Having the drain valve 1533 be coupled to a straight sidearm of the fragmenting and/or fragment-removing system 1000 may promote aspects herein such as promoting passive drainage when the drain valve 1533 is in a first position.

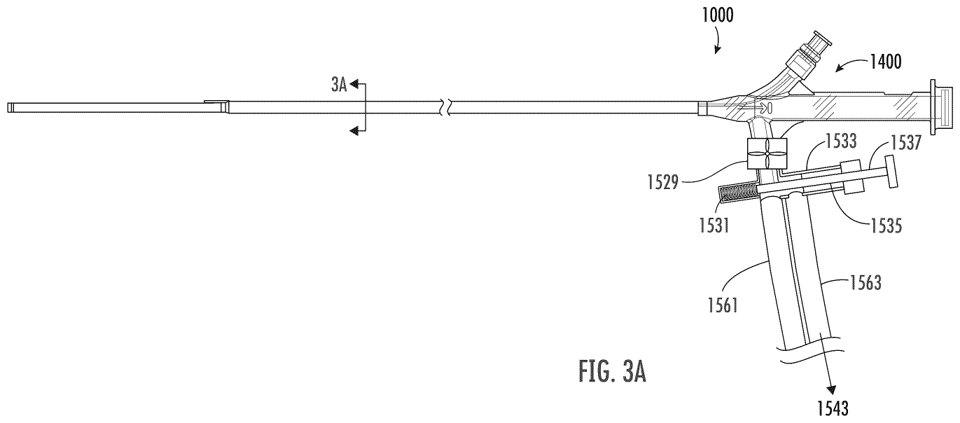

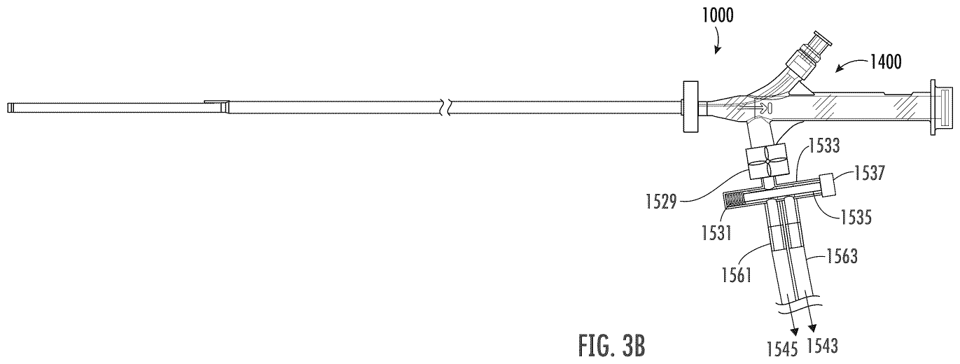

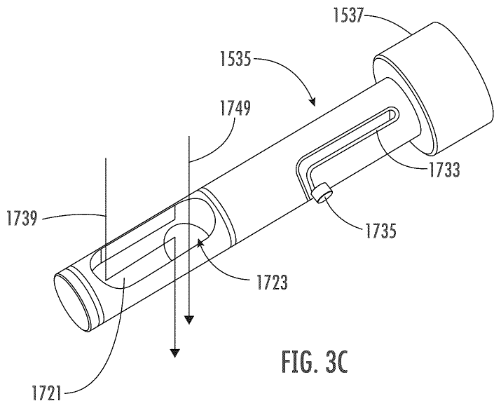

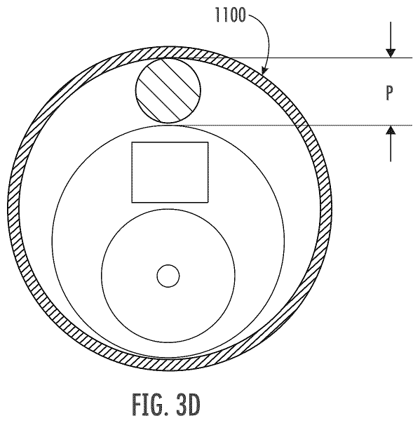

The drain valve 1533 may couple the outlet channel of the fragmenting and/or fragment-removing system 1000 to a drain conduit. The drain valve 1533 is configured to passively drain material from the outlet channel to the drain conduit when the movable member is in the first position, as illustrated in FIG. 2A and FIG. 3A. Additionally, the drain valve 1533 is configured to actively drain material from the outlet channel to the drain conduit when the movable member is in the second position, for instance as illustrated in FIG. 2B and FIG. 3B. That is, the drain valve 1533 is a movable member 1535 that is configured to move between a first position and a second position. In some embodiments, the movable member 1535 is a plunger such as an elongate plunger that extends in the same direction as the longitudinal direction or longitudinal axis of the fragmenting and/or fragment-removing system 1000, as illustrated in FIGS. 2A-2B and 3A-3B. For instance, the movable member 1535 may be a plunger that extends along and is configured to move in a longitudinal direction.

In some embodiments, the drain valve 1533 may include a dispositioning mechanism 1532 to disposition the movable member to the first position. The dispositioning mechanism 1532 can be a spring or other type of dispositioning mechanism. For instance, the drain valve 1533 can include a spring to disposition the movable member 1535 to a first position, as illustrated in FIG. 2A.

In some embodiments, the drain valve 1533 may include the visual flow indicator 1529. As illustrated in FIG. 2A, the visual flow indicator 1529 may be located between the outlet channel and the drain conduit such as the drain conduit 1541. Accordingly, visual flow indicator 1529 may be configured to indicate a flow rate of the material between the outlet channel and the drain conduit. In some embodiments, the visual flow indicator 1529 includes a paddlewheel 1599, as illustrated in FIG. 2A. Rotation of the paddlewheel 1599 may readily represent an amount of material flowing through the drain valve 1533.

In some embodiments, the drain conduit such as the drain conduit 1541 may be an individual drain conduit that is coupled to a suction source (e.g., suction source 1010). In such embodiments, the drain conduit 1541 may be an individual drain conduit configured to passively drain material 1543 from the outlet channel to the drain conduit 1541 when the movable member is in the first position, as illustrated in FIG. 2A, and actively drain material 1545 from the outlet channel to the drain conduit 1541 when the movable member is in the second position, as illustrated in FIG. 2A.

In some embodiments, the movable member 1535 may be a plunger extending along an inner surface of the body of the valve. The plunger may have a width (in the longitudinal direction) that is equal to or greater than a width of the vent 1539. As such, the plunger may be configured to expose the vent when the plunger is in the first position and may be configured to block at least a portion of the vent when the plunger is in the second position. For instance, when in the first position a portion of the movable member 1535 may expose a vent 1539 extending through a body of the drain valve 1533. As such, the movable member 1535 may permit fluidic communication via the vent 1539 between a lumen of the drain valve 1533 and an external environment around the drain valve 1533. This fluidic communication can mitigate the pressure imparted by the suction source on the fragmenting and/or fragment-removing system 1000 and thereby permit passive drainage of material 1543 when the movable member 1535 is in the first position, as illustrated in FIG. 2A.

Conversely, when in the second position a portion of the movable member 1535 may block (e.g., entirely block fluidic flow through) a vent 1539 extending through a body of the drain valve 1533. As such, the movable member 1535 may block fluidic communication via the vent 1539 between a lumen of the drain valve 1533 and an external environment around the drain valve 1533. Blocking the fluidic communication can cause the negative pressure (e.g., suction) to remain imparted by the suction source on the fragmenting and/or fragment-removing system 1000 and thereby permit active drainage 1545 when the movable member 1535 is in the second position, as illustrated in FIG. 2B. For instance, the movable member 1535 may move from the first position (e.g., a default position) to the second position responsive to activation of button 1537 located on an end of the movable member 1535. In some embodiments, a retention mechanism (not illustrated) such as a slot and corresponding pin (e.g., a pogo pin) or other type of mechanism can be configured to retain the movable member 1535 in the second position.

FIG. 3A illustrates an elevational view of an alternate arrangement of elements of a fragmenting and/or fragment-removing system with a drain valve in a first position in accordance with aspects of the present disclosure. FIG. 3B illustrates an elevational view of an alternate arrangement of elements of a fragmenting and/or fragment-removing system with a drain valve in a second position in accordance with aspects of the present disclosure. FIG. 3C illustrates a perspective view of an example of a movable member in FIG. 3A and FIG. 3B. As illustrated in FIG. 3A and FIG. 3B, an example of an embodiment of a fragmenting and/or fragment-removing system 1000 may be substantially the same as the fragmenting and/or fragment-removing system 1000 illustrated in FIG. 2A and FIG. 2B, respectively, other than the individual drain conduit 1541 been replaced with two separate drain conduits in the form of an active drain conduit 1561 and a passive drain conduit 1563 and the vent 1539 is omitted. That is, the drain conduit in the fragmenting and/or fragment-removing system 1000 illustrated in FIG. 3A and FIG. 3B includes the passive drain conduit 1563 and the active drain conduit 1561. The active drain conduit 1561 is couplable (e.g., is coupled to) a suction source such as the suction source 1010 and therefore is configured to operate at a reduced pressure relative to atmospheric pressure. The passive drain conduit 1563 is configured to operate at atmospheric pressure. For instance, the passive drain conduit 1563 may not be configured to be coupled to a suction source but rather may be coupled to a reservoir which is configured to operate at atmospheric pressure.

As illustrated in FIG. 3C, the drain valve 1533 may include a slot 1721. The slot 1721 may extend in a longitudinal direction. The slot 1721 may be configured to receive material from the outlet channel and may include a surface including an outlet 1723 extending therethrough. In some embodiments, the movable member 1535 may be a plunger configured to block an active drain path 1739 extending through the active drain conduit 1561 when the plunger is in a first position, as illustrated in FIG. 3A and may be configured to align the outlet 1723 with the active drain conduit 1561 when the movable member is in a second position. In some embodiments, the movable member 1535 may be a plunger configured to maintain a passive drain path 1749 in an open configuration when a movable member 1535 such as the plunger is in a first position and a closed configuration when the movable member 1535 is in a second position. The movable member 1535 may include locking mechanism such as a channel 1733 and a pin 1735 disposed within the channel 1733. The locking mechanism can be configured to lock the movable member 1535 in a given position such as the first position or the second position, as described herein.

FIG. 3D illustrates an end view along line 3A-3A in FIG. 3A. The end view in FIG. 3D is analogous to the end view in FIG. 1B, but is without the presence of the outer sheath 1300. That is, in some embodiments, the fragmenting and/or fragment-removing systems herein may employ an individual sheath (e.g., sheath 1000) defining a single lumen therethrough and having an inner surface and an outer surface, as illustrated in FIG. 3D.

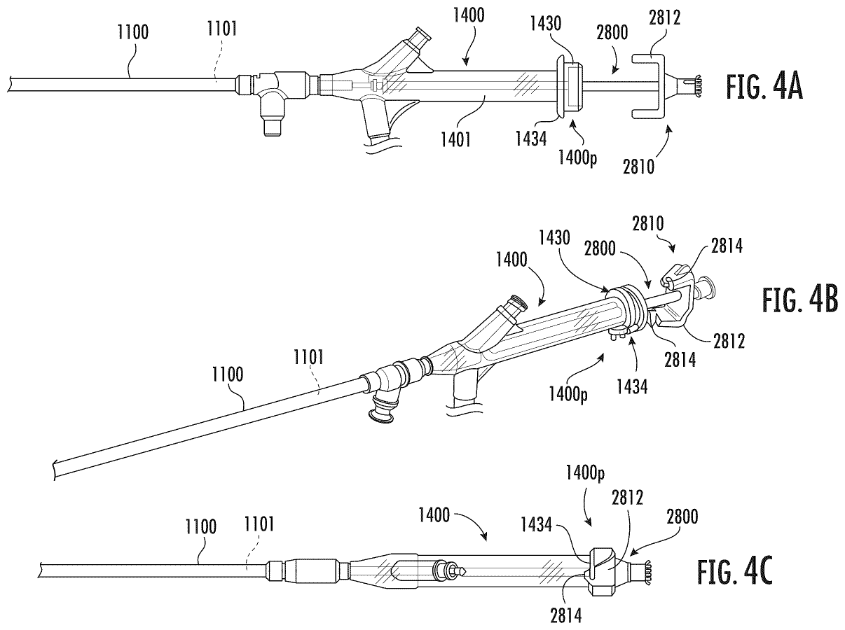

In some aspects, a dilator may be used to facilitate navigation of the distal end 1000d of a fragmenting and/or fragment-removing system 1000 formed in accordance with various principles of the present disclosure, and entry of the distal end 1000d with respect to the treatment site T. For instance, a dilator may be particularly useful in navigating through a generally fragile ureter and/or urethra and into a renal pelvis. In some aspects, a dilator may facilitate insertion of the fragment-removing system 1000 into a patient. In some aspects, at least the distal end (and optionally a portion or the full longitudinal extent) of the dilator may be covered with a hydrophilic and/or friction-reducing coating to facilitate insertion. In addition to, or instead of, facilitating access of a biasing member 1700 to a fragmenting and/or fragment-removing system 1000 formed in accordance with various principles of the present disclosure, the second sidearm 1420 of the body 1400 of the fragmenting and/or fragment-removing system 1000 may be used to facilitate insertion of a dilator with respect to the fragmenting and/or fragment-removing system 1000. In some aspects, the dilator includes a dilator hub which can be releasably engaged (e.g., screw tightened, etc.) with respect to a sidearm such as the second sidearm 1420 of the body 1400 can be inserted into lumen of a sheath such as the inner sheath 1100 via the proximal end 1400p of the body 1400, such as illustrated in FIG. 4A, FIG. 4B, and FIG. 4C.

More particularly, the example of an embodiment of a dilator 2800 illustrated in FIG. 4A, FIG. 4B, and FIG. 4C is inserted into the proximal end 1400p of the body 1400 which opens into the lumen 1401 of the body 1400 and into the lumen 1101 of the inner sheath 1100. In some aspects, the dilator 2800 may be coupled with the body 1400, such as to be maintained in place with respect to the inner sheath 1100. For instance, in the example of an embodiment illustrated in FIG. 4A, FIG. 4B, and FIG. 4C, the dilator 2800 may have a hub 2810 with one or more distal extensions 2812 configured to engage seal 1430 at the proximal end 1400p of the body 1400. In some aspects, the distal extensions 2812 include hooks 2814 configured to engage and to hook onto corresponding tabs 1434 on the seal 1430 to maintain the dilator 2800 in place with respect to the body 1400.

It will be appreciated that various features of one example of an embodiment of a medical device described herein may be present in others of the examples of embodiments described herein, whether or not explicitly stated or described. For purposes of clarity, not all components having the same reference number are numbered. It will further be appreciated that the various features described herein may be used singly or in any combination thereof. Therefore, the present invention is not limited to only the combination of features described with respect to the embodiments specifically described herein. Moreover, it will be appreciated that various operations of an above-described device or system may be performed by others of the above-described devices or systems.

Although embodiments of the present disclosure may be described with specific reference to medical devices and systems and procedures for treating the urinary tract, it should be appreciated that such medical devices and methods may be used to treat tissues of the gastrointestinal system, abdominal cavity, digestive system, urinary tract, reproductive tract, respiratory system, cardiovascular system, circulatory system, and the like. Moreover, the medical devices, instruments, tools, etc. of the present disclosure are not limited, and may include a variety of medical devices, instruments, tools, etc., for accessing body passageways, including, for example, ureteroscopes, duodenoscopes, catheters, bronchoscopes, colonoscopes, arthroscopes, cholangioscopes, cystoscopes, hysteroscopes, and the like. Such devices, instruments, tools, etc., may be disposable and for single use only, or sterilizable and reusable, the present disclosure not being limited in this regard.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A medical device, comprising:
a sheath defining a lumen therethrough and having an inner surface and an outer surface;
an outlet channel in a portion of the lumen; and
a drain valve configured to couple the outlet channel to a drain conduit, wherein the drain valve includes a movable member that is configured to move between a first position and a second position, and wherein the drain valve is configured to:
passively drain material from the outlet channel to the drain conduit when the movable member is in the first position; and
actively drain material from the outlet channel to the drain conduit when the movable member is in the second position.

2. The medical device of claim 1, wherein the drain valve is coupled to a sidearm extending from a body of the medical device.

3. The medical device of claim 2, wherein the sidearm is a straight sidearm.

4. The medical device of claim 1, wherein the drain valve includes a dispositioning mechanism to disposition the movable member to the first position.

5. The medical device of claim 4, wherein the dispositioning mechanism is a spring.

6. The medical device of claim 1, wherein the drain valve includes a visual flow indicator between the outlet channel and the drain conduit that is configured to indicate a flow rate of the material between the outlet channel and the drain conduit.

7. The medical device of claim 6, wherein the visual flow indicator includes a paddlewheel.

8. The medical device of claim 7, wherein the drain conduit is an individual drain conduit that is coupled to a suction source.

9. The medical device of claim 8, wherein the drain valve includes a vent.

10. The medical device of claim 9, wherein the movable member includes a movable cover disposed on an outer surface of drain valve, wherein the cover is configured to:
expose the vent when the cover is in the first position; and
block at least a portion of the vent when the cover is in the second position.

11. The medical device of claim 9, wherein the movable member is a plunger configured to:
expose the vent when the plunger is in the first position; and
block at least a portion of the vent when the plunger is in the second position.

12. The medical device of claim 1, wherein:
the drain conduit includes a passive drain conduit and an active drain conduit;
the active drain conduit is coupled to a suction source and is configured to operate at a reduced pressure relative to atmospheric pressure; and
the passive drain conduit is configured to operate at atmospheric pressure.

13. The medical device of claim 12, wherein the drain valve is configured to actively drain the material, via the active drain conduit, at the reduced pressure when the movable member is in the second position.

14. The medical device of claim 12, wherein the drain valve is configured to passively drain the material, via the passive drain conduit, at atmospheric pressure when the movable member is in the first position.

15. The medical device of claim 12, wherein the drain valve includes a slot, wherein the slot is configured to receive the material from the outlet channel, the slot having a surface including an outlet, wherein the movable member is a plunger configured to:
block an active drain path extending through the active drain conduit when the movable member is in a first position; and
align the outlet with the active drain conduit when the movable member is in a second position.

16. A system for removing material from an anatomical site, comprising:
a sheath defining a single lumen therethrough and having an inner surface and an outer surface;
a tubular elongate member extending through the single lumen and defining a distal inlet channel therethrough fluidly couplable with a fluid source to irrigate the anatomical site;
an outlet channel between the inner surface of the sheath and the outer surface of the tubular elongate member, wherein the outlet channel is couplable with a suction source to aspirate material from the anatomical site; and
a drain valve configured to couple the outlet channel to a drain conduit, wherein the drain valve includes a movable member that is configured to move between a first position and a second position, and wherein the drain valve is configured to:
passively drain material from the outlet channel to the drain conduit when the movable member is in the first position; and
actively drain material from the outlet channel to the drain conduit when the movable member is in the second position.

17. The system of claim 16, further comprising a fragmenting device that extends through the tubular elongate member.

18. The system of claim 17, wherein the material comprises a bodily mass or fragments thereof.

19. A system for removing material from an anatomical site, comprising:
an outer sheath defining an outer lumen therethrough and having an inner surface and an outer surface;
an inner sheath defining an inner lumen therethrough and having an inner surface and an outer surface;
a tubular elongate member extending through the inner lumen and defining a distal inlet channel therethrough fluidly couplable with a fluid source to irrigate the anatomical site;
a biasing member configured to maintain an outlet channel between the inner surface of the inner sheath and the outer surface of the tubular elongate member, wherein the outlet channel is couplable with a suction source to aspirate material from the anatomical site;
a drain valve configured to couple the outlet channel to a drain conduit, wherein the drain valve includes a movable member that is configured to move between a first position and a second position, and wherein the drain valve is configured to:
passively drain material from the outlet channel to the drain conduit when the movable member is in the first position; and
actively drain material from the outlet channel to the drain conduit when the movable member is in the second position; and
a visual flow indicator located between the outlet channel and the drain conduit, the visual flow indicator configured to indicate a flow rate of the material between the outlet channel and the drain conduit.

20. The system of claim 19, further comprising:
a dilator insertable within at least one of the outer sheath or the inner sheath; and a sensor operatively associated with one of the inner sheath or the tubular elongate member, or insertable separate from the inner sheath and the tubular elongate member.
